(19) United States
(12) Patent Application Publication
(10) Pub. No.: US 2007/0082856 A1
(43) Pub. Date: Apr. 12, 2007

(54) Combination therapy comprising the use of et-743 and doxorubicin for treating cancer

(75) Inventors: Luca Gianni, Milan (IT); Maurizio D'Incalci, Milan (IT); Filippo de Braud, Milan (IT); Silvia Marsoni, Milan (IT); Jose Maria Jimeno Donaque, Madrid (ES); Luis Lopez Lazaro, Madrid (ES)

Correspondence Address: KING & SPALDING, 1185 AVENUE OF THE AMERICAS, NEW YORK, NY, 10036-4003, US

(73) Assignee: Pharma Mar, S.A.U., Madrid (ES)

(21) Appl. No.: 10/579,251

(22) PCT Filed: Nov. 12, 2004
(86) PCT No.: PCT/GB04/50025

§ 371 Date: Oct. 20, 2006

(30) Foreign Application Priority Data
Nov. 14, 2003 (GB) 0326486.8

(51) Int. Cl. A61K 31/704

(52) U.S. Cl. 514/34

(57) ABSTRACT

Methods of treating a human body for cancer are provided. In one aspect, a therapeutic amount of doxorubicin is administered in combination with ET-743 in a dose range between 0.5 and 1 mg/m². In a related aspect, an effective therapeutic amount of ET-743 is administered in combination with doxorubicin in a dose range between 40 and 80 mg/m².

The invention relates to a combination of treatments, more particularly a combination treatment for cancer.

### FIELD OF THE INVENTION

The present invention is directed to the use of ecteinascidin 743 for cancer therapy, in particular to the use of ecteinascidin 743 in combination with another active drug, doxorubicin, for the treatment of cancer.

### BACKGROUND OF THE INVENTION

Cancer comprises a group of malignant neoplasms that can be divided into two categories, carcinoma, comprising a majority of the cases observed in the clinics, and other less frequent cancers, which include leukemia, lymphoma, central nervous system tumors and sarcoma. Carcinomas have their origin in epithelial tissues while sarcomas develop from connective tissues and those structures that had their origin in mesoderm tissues. Sarcomas can affect, for instance, muscle or bone and occur in the bones, bladder, kidneys, liver, lung, parotid, spleen, etc.

Cancer is invasive and tends to metastasise to new sites. It spreads directly into surrounding tissues and also may be disseminated through the lymphatic and circulatory systems.

Many treatments are available for cancer, including surgery and radiation for localised disease, and drugs. However, the efficacy of available treatments on many cancer types is limited, and new, improved forms of treatment showing clinical benefit are needed.

This is especially true for those patients presenting with advanced and/or metastatic disease. It is also true for patients relapsing with progressive disease after having been previously treated with established therapies for which further treatment with the same therapy is mostly ineffective due to acquisition of resistance or to limitations in the administration of the therapies due to associated toxicities,

Chemotherapy plays a significant part in cancer treatment, as it is required for treatment of advanced cancers with distant metastasis and often helpful for tumor reduction before surgery. Many anti-cancer drugs have been developed based on various modes of action.

The most commonly used types of anticancer agents include: DNA-alkylating agents (for example, cyclophosphamide, ifosfamide), antimetabolites (for example, methotrexate, a folate antagonist, and 5-fluorouracil, a pyrimidine antagonist), microtubule disrupters (for example, vincristine, vinblastine, paclitaxel), DNA intercalators (for example, doxorubicin, daunomycin, cisplatin), and hormone therapy (for example, tamoxifen, flutamide). The ideal antineoplastic drug would kill cancer cells selectively, with a wide therapeutic index relative to its toxicity towards non-malignant cells. It would also retain its efficacy against malignant cells, even after prolonged exposure to the drug.

Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents.

The ecteinascidins (herein abbreviated ETs) are exceedingly potent antitumor agents isolated from the marine tunicate Ecteinascidia turbinata. Several ecteinascidins have been reported previously in the patent and scientific literature. See, for example U.S. Pat. No. 5,089,273, which describes novel compounds extracted from the tropical marine invertebrate, Ecteinascidia turbinata, and designated therein as ecteinascidins 729, 743, 745, 759A, 759B and 770. These compounds are useful as antibacterial and/or antitumor agents in mammals. U.S. Pat. No. 5,478,932 describes ecteinascidins isolated from the Caribbean tunicate Ecteinascidia turbinata, which provide in vivo protection against P388 lymphoma, B16 melanoma, M5076 ovarian sarcoma, Lewis lung carcinoma, and the LX-1 human lung and MX-1 human mammary carcinoma xenografts.

One of the ETs, ecteinascidin-743 (ET-743), is a novel tetrahydroisoquinoline alkaloid with considerable antitumor activity in murine and human tumors in vitro and in vivo, and is presently in clinical trials. ET-743 possesses potent antineoplastic activity against a variety of human tumor xenografts grown in athymic mice, including melanoma and ovarian and breast carcinoma.

A clinical development program of ET-743 in cancer patients was started with phase I studies investigating 1-hour, 3-hour, 24-hour and 72-hour intravenous infusion schedules and a 1 hour daily×5 (d×5) schedule. Promising responses were observed in patients with sarcoma and breast and ovarian carcinoma. Therefore this new drug is currently under intense investigation in several phase II clinical trials in cancer patients with a variety of neoplastic diseases.

Further detail on the use of ET-743 for the treatment of the human body for cancer is given in WO 0069441, incorporated herein by reference in its entirety. At pages 8 and 9, this patent specification indicates that ET-743 may be employed in a combination therapy with another drug. A list of candidates for the other drug is given, and mentions doxorubicin.

A recent review of ET-743, its chemistry, mechanism of action and preclinical and clinical development can be found in van Kesteren, Ch. et al., 2003, Anti-Cancer Drugs, 14 (7), pages 487-502: “Yondelis (trabectedin, ET-743): the development of an anticancer agent of marine origin”, and references therein.

Combination therapy using drugs with different mechanisms of action is an accepted method of treatment which helps prevent development of resistance by the treated tumor. In vitro activity of ET-743 in combination with other anticancer agents has been studied, see for example WO 02 36135, incorporated herein by reference in its entirety. In particular, WO 0236135 mentions the combination of ET-743 with doxorubicin. A synergistic effect is noted in tests on animal models.

Meco et al. report on “Effective combination of ET-743 and doxorubicin in sarcoma: preclinical studies” in Cancer Chemother Pharmacol (2003) 52: 131-138. The combination was tested against a sarcoma cell line and against mice with transplanted human sarcomas. They report an additive effect and suggest that the combination might be effective for tumors displaying low sensitivity to each drug given alone.

It is an object of the invention to provide an efficacious combination treatment of cancer based on ET-743 with doxorubicin.

### SUMMARY OF THE INVENTION

According to the present invention, we provide a combination therapy for the treatment of cancer which employs ecteinascidin 743 and doxorubicin, using a cyclical dosing protocol. Typical dosing protocols for the combination therapy are provided. From phase I clinical trials, we have determined that a combination of ET-743 and doxorubicin is tolerable and feasible, with evidence of antitumor activity.

We also provide a method of treating a cancer patient, which comprises administering ET-743 and doxorubicin. The ET-743 and doxorubicin are preferably administered on the same day of a predetermined cycle.

We further provide the use of ET-743 in the preparation of a medicament for carrying out the method of treatment. We also provide the use of the doxorubicin, in the preparation of a medicament for carrying out the method of treatment. We provide the use of ET-743 and the doxorubicin, in the preparation of a medicament for carrying out the method of treatment.

### DETAILED DESCRIPTION

ET-743 is a natural compound represented by the following formula:

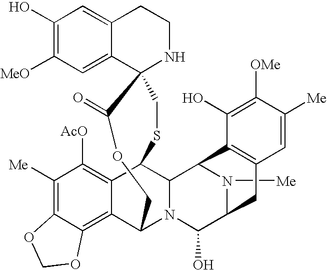

As used herein, the term “ET-743” extends to natural and synthetic ET-743 and also covers any pharmaceutically acceptable salt, ester, solvate, hydrate or a prodrug compound which, upon administration to the recipient is capable of providing (directly or indirectly) the compound ET-743. The preparation of salts and other derivatives, and prodrugs, can be carried out by methods known in the art.

ET-743 is typically supplied and stored as a sterile lyophilized product, with ET-743 and excipient in a formulation adequate for therapeutic use, in particular a formulation containing mannitol and a phosphate salt buffered to an adequate pH.

It is currently preferred to administer the ET-743 by infusion. The infusing step is typically repeated on a cyclic basis, which may be repeated as appropriate over for instance 1 to 35 cycles. The cycle includes a phase of infusing ET-743, and usually also a phase of not infusing ET-743. Typically the cycle is worked out in weeks, and thus the cycle normally comprises one or more weeks of an ET-743 infusion phase, and one or more weeks to complete the cycle. In one embodiment a cycle of 3 weeks is preferred. Alternatively it can be from 2 to 6 weeks. The infusion phase can itself be a single administration in each cycle of say 1 to 72 hours, more usually 1, 3 or 24 hours, or infusion on a daily basis in the infusion phase of the cycle for preferably 1 to 5 hours, especially 1 or 3 hours. Thus, for example, the ET-743 might be administered on each of the first five days of a 3 week cycle. We currently prefer a single administration at the start of each cycle or two administrations in each cycle, for instance, on days 1 and 8 every 21 days.

The dose will be selected according to the dosing schedule, having regard to the existing data on Dose Limiting Toxicity, on which see for example the incorporated WO patent specifications, and also see van Kesteren, Ch. et al., 2003, Anti-Cancer Drugs, 14 (7), pages 487-502: “Yondelis (trabectedin, ET-743): The development of an anticancer agent of marine origin”. This article is incorporated herein in full by specific reference.

For a single administration of ET-743 at the start of each cycle or twice per cycle, we prefer a dose in the range 0.2 to 2 mg/m², more preferably 0.4 to 1.5 mg/m², most preferably 0.5 to 1.2 mg/m². For this combination we particularly prefer a dose from below 0.8 mg/m², more preferably from about 0.2 to about 0.775 mg/m², most preferably about 0.5 to about 0.75 mg/m². Particularly preferred is a dose about 0.6 or about 0.7 mg/m².

As noted in the incorporated article by van Kesteren, the combination of ET-743 with dexamethasone gives unexpected advantages. It has a role in hepatic prophylaxis. We therefore prefer to administer dexamethasone to the patient, typically at around the time of infusing the ET-743. For example, we prefer to give dexamethasone before ET-743 on the same day, The administration of dexamethasone can be extended, for example to one or more days preceding or following ET-743.

The ET-743 is administered as part of a combination therapy with doxorubicin.

Doxorubicin is indicated for the treatment of many cancers, including for instance breast cancer, ovarian cancer, transitional cell bladder cancer, bronchogenic lung cancer, thyroid cancer, gastric cancer, soft tissue and osteogenic sarcomas, neuroblastoma, Wilms' tumor, malignant lymphoma (Hodgkin's and non-Hodgkin's), acute myeloblastic leukemia, acute lymphoblastic leukemia, Kaposi's sarcoma related to acquired immunodeficiency syndrome (AIDS).

In one embodiment of the invention, the doxorubicin does not take the form of doxorubicin in pegylated liposomal form, such as that commercially available under the trade mark Doxil.

For the present invention, the doxorubicin is preferably administered by intravenous push as part of the cycle of treating the patient. The doxorubicin is suitably in the form of a pharmaceutically acceptable salt, such as the hydrochloride. In common with other usage, the term “doxorubicin” in this specification includes salts of doxorubicin,

We prefer that the doxorubicin is given on the same day as ET-743, either before or after. An interval between the two drugs may be necessary, an interval of about 1 hour is preferred. For a cycle of 3 weeks, we prefer administration on day 1 with ET-743. Other administration protocols can be designed having regard to this embodiment.

The dosage amount of doxorubicin is preferably in the range from 30 to 100 mg/m²/day, more preferably 40 to 80 mg/m²/day. At this stage, we currently prefer a dose of about 50 mg/m²/day or about 60 mg/m²/day. Infusion times for doxorubicin are generally up to 6 hours, more preferable 1-3 hours, with 1 hour most preferred.

Depending on the type of tumor and the developmental stage of the disease, the treatments of the invention are useful in preventing the risk of developing tumors, in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the combination therapy is used according to the above schedules and dosages for the treatment of sarcoma, osteosarcoma, ovarian cancer, breast cancer, melanoma, colorectal cancer, mesothelioma, renal cancer, endometrial cancer and lung cancer. Most preferably the patients are sarcoma patients, especially those with a soft tissue sarcoma and breast cancer.

In a further aspect of the present invention, a medical kit for administering ET-743 in combination with doxorubicin is provided, comprising printed instructions for administering ET-743 according to the dosing schedules set forth above, and a supply of ET-743 in dosage units for at least one cycle, wherein each dosage unit contains the appropriate amount of ET-743 for the treatments as defined above and a pharmaceutically acceptable carrier.

Although guidance for the dosage is given above, the correct dosage of the compounds will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

### EXAMPLE

### Example 1

### Phase I Clinical Trial

The objective of this study was the definition of the least toxic sequence (LTS) and optimal therapeutic dose of ET-743 in combination with doxorubicin (doxo) in patients with untreated metastatic soft tissue sarcomas (STS) and advanced pre-treated anthracyline-naïve breast cancer patients (ABC).

In this multicenter dose and LTS finding trial, patients were assigned consecutively to start either with sequence A (ET-743 before Doxo) or with the reverse sequence (B) at the following dose levels every 21 days:

| ET-743 | Doxorubicin |
|---|---|
| 600 μg/m² | 60 mg/m² |
| 700 μg/m² | 60 mg/m² |
| 800 μg/m² | 60 mg/m² |

Pharmacokinetic [PK] of both drugs was determined for the 2 sequences at cycle 1 and cycle 2, when patients received the drugs in the reverse order of administration. Alternating sequence was discontinued at observation of dose limiting toxicity [DLT]: observation of grade 4 hematological toxicity for more than 3 days at the entry level. Both drugs were administered on day 1, with a 1 h interval between the 2 drugs (ET-743, 3-hr infusion i.v. and Doxo, 1-hr infusion i.v push with steroids & 5-HT₃ antagonists as antiemetic prophylaxis). Oral steroids premedication for ET-743 was given 24 h before and for 48 h following the day of treatment. Doxo was administered at the fixed dose of 60 mg/m², while ET-743 was started at 600 μg/m² and escalated thereafter in subsequent cohorts of at least 3 new cases. Patients continued treatment until progressive disease (PD) or intolerance, and were restaged every 2 cycles for activity.

In this study, 22 patients were enrolled and evaluable. The patients were required to have normal liver, renal, cardiac and haematologic functions and good performance status for entry into the study. Enrolment was restricted to breast cancer and soft tissue sarcoma. Limitations on prior chemotherapy were also applied: prior adjuvant therapy was permitted if recurrence≧6 months from end and receiving maximum cumulative Doxo-equivalent dose≦280 mg/m². Table 1 shows the patients and study characteristics.

TABLE 1

| | | |
|---|---|---|
| Patients entered/evaluable | 23/22 | |
| Patients age median (yrs) (range) | 52 | (38-75) |
| Sex M/F | 3/19 | |
| Performance Status | | |
| ECOG 0 | 91% | |
| ECOG 1 | 9% | |
| Tumor type | | |
| Advanced Breast Cancer (ABC) | 4 | |
| Soft Tissue Sarcoma (STS) | 18 | |
| Dose Level ET-743 (sequence A/B) | | |
| 600 (all STS) | 10 | (6A/4B) |
| 700 (all STS) | 3 | (1A/2B) |
| 800 (5 STS/4ABC) | 9 | (6A/3B) |

| | |
|---|---|
| Prior therapy (2 cases) | 1 STS pt at dose level 600 |
| | (6 cycles epirubicin as adjuvant) |
| | 1 STS pt at dose level 800 |
| | (6 cycles doxorubicin as |
| | neoadjuvant) |

First cycle dose limiting toxicities (DLT) were defined as
a) Grade 4 absolute neutrophil count (ANC) during more than 7 days
b) Febrile neutropenia
c) Grade 4 platelets or haemoglobin (Hb)
d) Grade 3 stomatitis during 3 days or more days
e) Hepatic: Elevation of alkaline phosphatase (AlkPhos)≧G3 and elevation of bilirubin/transaminases/AlkPhos of any grade with or without recovery by day 28

Table 2 shows the dose escalation levels and accruals to each level and the DLTs experienced at each dose level.

TABLE 2

| | | |
|---|---|---|
| A | DOSE ET-743 | B |

| No. patients | DLT | No. patients | No. patients | DLT |
|---|---|---|---|---|
| 6 | 0 | 600 | 4 | 0 |
| | | 10 | | |
| 1 | 0 | 700 | 2 | 0 |
| | | 3 | | |
| 6 | 2 | 800 | 3 | 2 |
| | | 9 | | |

No DLTs had been noted among the pts enrolled up to 700 μg/m². The dose was escalated to 800 μg/m² at which 4 DLTS, (2 in sequence A due to grade 4 ANC>7 days and febrile neutropenia, and the other 2 in sequence B with ANC grade 4>7 days plus G3 asthenia and febrile neutropenia). Comparison of the plasma disposition of ET-743 and doxorubicin in patients receiving both sequences did not reveal any significant pharmacokinetic interaction.

Antitumor activity was observed: 5 pts had a confirmed partial response (PR) (2 at ET-743 dose level 600 μg/m², 1 at ET-743 dose level 700 μg/m² and 2 at ET-743 dose level 800 μg/m²) and 5 a long lasting (>6 months) stable disease (SD) (2 at ET-743 dose level 600 μg/m², 1 at ET-743 dose level 700 μg/m² and 2 at ET-743 dose level 800 μg/m²). Table 3 shows the antitumor activity data.

TABLE 3

| | | ET-743 | | | |
|---|---|---|---|---|---|
| | PRIMARY | DOSE | SITES OF | BEST | TTP |
| PT # | TUMOR TYPE | μg/m² | DISEASE | RESP | months |
| 3 | STS | 600 | LN, lung, bone | PR | 5 |
| 9 | STS-ovary | 600 | Abdo, skin | PR | 5 |
| 5 | STS | 600 | Pelvis, bone | SD | 12 |
| 10 | STS | 600 | Lung | SD | 5 |
| 13 | STS | 700 | Lung, | PR | 7+ |
| | | | mediastinum, LN | | |
| 11 | STS-uterus | 700 | Lung | SD | 6+ |
| 15 | STS-uterus | 800 | Abdomen, pelvis | PR | 4 |
| | (prior | | | | |
| | adjuvant | | | | |
| | doxo-6 cycles) | | | | |
| 16 | ABC | 800 | Pleura, chest wall, | PR | 5+ |
| | | | LN | | |
| 18 | STS-cervix | 800 | Lung | SD | 6+ |
| 21 | STS | 800 | Lung, | SD | 4+ |
| | | | subcutaneous | | |

TTP (Time to Progression)

For the purposes of this study, the Maximum Tolerated Dose (MTD) was reached when out of 6 patients 2 experienced DLTs.

The MTD was defined by prolonged grade 4 neutropenia/febrile neutropenia at 800 μg/m² of ET-743 and 60 mg/m² of Doxo. The most relevant non-haematologic toxicity was the reversible alteration of transaminases at the higher doses after multiple cycles, Grade 4 neutropenia at the first dose level nullified the application of alternating sequence A and B in the same patients. Toxicity was similar with both sequences and order of administration did not influence the pharmacokinetics of either drug. Antitumor activity was observed at 600-700 μg/m² of ET-743 in combination with Doxo.

### Example 2

### Phase I Clinical Trial

Another phase I study was performed with the combination of ET-743 and doxorubicin. The objective of this study was to determine the safety profile and the optimal therapeutic dose of ET-743 in combination with doxorubicin (doxo) in patients with advanced gynecology and breast cancer and sarcoma.

Six dose levels of ET-743 were explored in the dose escalation phase of the study (300, 400, 500, 600, 700 and 800 μg/m²), whereas doxorubicin was administered at the fixed dose of 50 mg/m². Doxo was administered by i.v. push and immediately followed by ET-743, that was administered by 3-hr infusion. Doxo was given on day 1 only, while ET-743 was given on days 1 and 8 of the cycle. The cycle was repeated every 21 days.

A cohort of 3 to 6 patients was treated at each dose level according to the type and degree of toxicities observed. The main inclusion criteria the following:

Advanced solid tumor (preferably of the following types: gynecological and breast cancer and soft tissue sarcoma)

Maximum cumulative dose of prior doxo≦300 mg/m² and of prior epirubicin≦540 mg/m²

ECOG performance status≦1

Normal liver, renal, cardiac and haematologic functions

In this study, 20 patients were enrolled and evaluable. Table 4 shows the patients accrual and dose escalation status.

TABLE 4

| Dose Level | | | | |
|---|---|---|---|---|
| ET-743 | Doxo | No. | No. | |
| (μg/m²) | (μg/m²) | Patients | DLTs | DLT Type |
| 300 | 50 | 3 | — | — |
| 400 | 50 | 3 | — | — |
| 500 | 50 | 3 | — | — |
| 600 | 50 | 3 | — | — |
| 700 | 50 | 5 | 1 | failure to administer day 8 |
| | | | | infusion due to liver function |
| | | | | tests increase |
| 800 | 50 | 2 | 2 | failure to administer day 8 |
| | | | | infusion due to G3 neutropenia |
| | | | | ANC <500 for more than 5 |
| | | | | days and PLT >25,000 |

At the end of this study the MTD was defined at 700 μg/m² of ET-743 and 50 mg/m² of Doxo.

1. A method of treating a human body for cancer comprising administering an effective therapeutic amount of doxorubicin, in combination with ET-743 in a dose range between 0.5 and 1 mg/m² for ET-743.

2. A method of treating a human body for cancer comprising administering an effective therapeutic amount of ET-743, in combination with doxorubicin in a dose range between 40 and 80 mg/m² for doxorubicin.

3. The method according to claim 1 or 2, wherein doxorubicin and ET-743 are provided as separate medicaments for administration at different times.

4. The method according to claim 3, wherein doxorubicin is administered prior to the administration of ET-743.

5. The method according to claim 4, wherein doxorubicin and ET-743 are administered by intravenous injection.

6. The method according to claim 5, wherein the infusion time for intravenous injection is up to 3 hours for doxorubicin and up to 24 hours for ET-743.

7. The method according to claim 6, wherein the infusion time for intravenous injection is about 1 hour for doxorubicin and about 3 hours for ET-743.

8. The method according to claim 7, where the infusions are carried out at an interval of 1 to 6 weeks.

9. The method according to claim 8, wherein the infusions of both drugs are carried out once every 21 days.

10. The method according to claim 8, wherein the infusion of doxorubicin is carried out on day 1 and the infusion of ET-743 on days 1 and 8, every 21 days.

11. The method according to claim 9 or 10, wherein doxorubicin is administered in a dosage of up to 60 mg/m², followed by ET-743 which is administered in a dosage of up to 0.7 mg/m².

12. The method according to claim 11, wherein doxorubicin is administered in a dosage about 60 mg/m², followed by ET-743 which is administered in a dosage about 0.7 mg/m².

13. The method according to claim 11, wherein doxorubicin is administered in a dosage about 50 mg/m², followed by ET-743 which is administered in a dosage about 0.6 mg/m².

14. A method according to any preceding claim, in which the patient has a cancer selected from sarcoma, osteosarcoma, ovarian cancer, breast cancer, melanoma, colorectal cancer, mesothelioma, renal cancer, endometrial cancer and lung cancer.

15. A method according to claim 14, in which the patient has a cancer selected from sarcoma, ovarian cancer, endometrial cancer and breast cancer.

16. The use of doxorubicin in the preparation of a medicament for a method according to any of claims 1 to 15.

17. The use of ET-743 in the preparation of a medicament for a method according to any of claims 1 to 15.

18. A medical kit for administering ET-743 in combination with doxorubicin, comprising a supply of ET-743 in dosage units for at least one cycle, wherein each dosage unit contains the appropriate amount of ET-743 for the treatments and a pharmaceutically acceptable carrier, and printed instructions for administering ET-743 according to a dosing schedule.
